(19) United States
(12) Patent Application Publication
(10) Pub. No.: US 2026/0283851 A1
(43) Pub. Date: Sep. 24, 2026

(54) FLEXIBLE WELDING HELMET

(71) Applicant: TECMEN ELECTRONICS CO., LTD, Nanjing, Jiangsu (CN)

(72) Inventors: Yali SI, Nanjing (CN); Pengpeng HU, Nanjing (CN)

(21) Appl. No.: 19/472,607

(22) PCT Filed: Dec. 8, 2023
(86) PCT No.: PCT/CN2023/137458

§ 371 Date: Oct. 3, 2025

(30) Foreign Application Priority Data
Apr. 4, 2023 (CN) 202310354075.9
Apr. 4, 2023 (CN) 202320732239.2

(51) Int. Cl. A61F 9/06

(52) U.S. Cl. CPC A61F 9/061

(57) ABSTRACT

The present application discloses a flexible welding helmet, comprising: a viewing window assembly; a flexible protection shield releasably connected to the viewing window assembly; and a belt connected to the flexible protection shield and having an adjustable length, wherein the viewing window assembly comprises: a distal housing, a proximal housing, and a flip member pivotally connected to the distal housing, the distal housing is provided with a contact edge, the proximal housing is provided with a contact edge and a proximal edge spaced from the contact edge, the flexible protection shield is partially clamped between the contact edge of the distal housing and the contact edge of the proximal housing, the proximal edge of the proximal housing has a curved contour which has a notch, the notch is configured to direct downwards when the flexible welding helmet is worn, and an eye protection assembly is releasably installed in the flip member.

### CROSS REFERENCE TO RELATED APPLICATIONS

This application is a 371 National Stage of International Application No. PCT/CN2023/137458, filed on Dec. 8, 2023, which claims priority to Chinese Patent Application No. 202310354075.9, filed on Apr. 4, 2023, and Chinese Patent Application No. 202320732239.2, filed on Apr. 4, 2023, the disclosures of which are herein incorporated by reference in their entirety.

### FIELD

The present application generally relates to a flexible welding helmet.

### BACKGROUND

Different than a conventional rigid welding helmet, a flexible welding helmet, especially its major part, is readily foldable for storage, can be worn by a user to accomplish welding works in a small space, and is easy to carry. Therefore, the flexible welding helmet is usually as a personal safety device for a welding operator often working in a small space.

At presents, there are a plurality of flexible welding helmets available in the market. Some flexible welding helmets have a flexible shielding material which is configured to cover only the face of a user for the sake of portability and lightweight. In the field welding operation, the user's head may be burnt out by welding arcs because the flexible shielding material is too small to cover the entire head of the user. Furthermore, when those flexible welding helmets are worn by the user, the flexible shielding material might be closer to the face of the user such that the user may breathe with difficulty or that the user may feel uncomfortable due to friction of his or her face with the flexible shielding material.

Another flexible welding helmets according to the prior art might be designed to have a flexible shielding material configured to substantially cover a user's head. However, the flexible shielding material of those flexible welding helmets is still too close to the face of the user who is wearing the flexible welding helmets on his or her head such that the user may breathe with difficulty or that the user may feel uncomfortable due to friction of his or her face with the flexible shielding material.

Another flexible welding helmets according to the prior art might be designed to have both of a headgear and a flexible shielding material. Although the existence of the headgear will not enable the flexible shielding material to be too close to the face of a user who is wearing the flexible welding helmets on his or her head, the flexible welding helmets will have to be made and assembled with excessive parts. Besides, because a viewing window of the flexible welding helmets is connected to the headgear, the viewing window might be too far away from eyes of the user who is wearing the flexible welding helmets on his or her head. In this case, the visual area of the viewing window through which the user can watch outsides by his or her eyes is negatively decreased.

### SUMMARY

In order to solve the above issues, the present application proposes a novel flexible welding helmet such that a user's head is reliably protectable by the flexible welding helmet, a viewing window of the flexible welding helmet can be as close as possible to eyes of the user who is wearing the flexible welding helmet on his or her head, and the user can breathe comfortably and smoothly when the flexible welding helmet is worn on his or her head.

According to an aspect of the present application, a flexible welding helmet is provided, which comprises:
a viewing window assembly;
a flexible protection shield releasably connected to the viewing window assembly; and
a belt connected to the flexible protection shield and having an adjustable length, wherein the viewing window assembly comprises:
   a distal housing, a proximal housing, and a flip member pivotally connected to the distal housing, the distal housing is provided with a contact edge, the proximal housing is provided with a contact edge and a proximal edge spaced from the contact edge, the flexible protection shield is partially clamped between the contact edge of the distal housing and the contact edge of the proximal housing, the proximal edge of the proximal housing has a curved contour formed with a notch, the notch is configured to direct downwards when the flexible welding helmet is worn. In the meanwhile, when the helmet is worn, the fact that the contact edge of the proximal housing is spaced from the proximal edge of the proximal housing enables the flexible protection shield not to be excessively close to a user's face, resulting in his or her comfortable feeling.

According to the present application, the notch of the curved contour of the proximal edge is configured such that when the flexible welding helmet is worn by a user on his or her head, the bridge of the user's nose or a part of the nose can be received within the space of the notch such that the viewing window assembly can be closer to the face of the user, especially his or her eyes. Because the viewing window assembly is closer to the eyes, the visual area of the viewing window assembly through which the user can watch outsides by his or her eyes can be enlarged. This will enable the user, who is wearing the welding helmet on his or her head, to reliably observe welding results at the welding site.

In an embodiment, the proximal edge of the proximal housing is curved C-shaped such that opposing left and right ends of the proximal edge are farther away from the flip member than the middle portion of the proximal edge, and the notch of the C-shape directs downwards when the flexible welding helmet is worn.

According to the present application, the curved C-shaped proximal edge can more suitably follow human facial contour such that when the flexible welding helmet is worn by a user on his or her head, the viewing window assembly can be closer to the user's face.

In an embodiment, a partition wall is formed between the contact edge of the proximal housing and the proximal edge of the proximal housing, and at least one aperture is formed in the partition wall.

According to the present application, the fact that the aperture is formed in the partition wall between the contact edge of the proximal housing and the proximal edge of the proximal housing enables air to smoothly flow between the welding helmet and a user's face after the flexible welding helmet is worn by the user.

In an embodiment, an eye shroud made of a soft material or a soft sweat-absorbent material is releasably installed on the proximal edge of the proximal housing, and the eye shroud is shaped to follow the proximal edge. In this way, when the flexible welding helmet is worn by a user on his or her head, the eye shroud, when contacting the user's face, will let him or her feel more comfortable such that he or she will be able to work for long hours with wearing the welding helmet on his or her head for his or her protection. Furthermore, the eye shroud can absorb human facial sweat, alleviating the dis-comfortable feeling of him or her working for long hours.

In an embodiment, the contact edge of the proximal housing and the contact edge of the distal housing are curved such that opposing left and right ends of the contact edges are farther away from the flip member than the middle portion of the contact edges. The contact edges are curved such that the viewing window assembly is more suitably adapted to a user's facial contour when it is connected to the flexible protection shield. This will enable the viewing window assembly to be closer to the user's face when the welding helmet is worn.

In an embodiment, the flexible protection shield has a part which locates under the viewing window assembly and which is configured to protrude distally or be planar when the flexible welding helmet is worn.

In an embodiment, an eye protection assembly is releasably installed in the flip member.

In an embodiment, the flip member is pivotable between a close position and a turned-up position, in which close position, the eye protection assembly covers a hollow interior space of the viewing window assembly defined by the distal housing and the proximal housing, and in which turned-up position, the eye protection assembly uncovers the hollow interior space.

In an embodiment, the flip member is formed with a side wall to define a space in which the eye protection assembly is installed.

In an embodiment, at least one pair of grooves are formed in the side wall of the flip member, the grooves of the pair are configured to oppose each other to receive bulges of a circlip installed in the space of the flip member. The circlip is configured to contact the eye protection assembly to secure it within the space of the clip member.

In an embodiment, the at least one pair of grooves include a pair of first grooves and a pair of second grooves, in the close position of the clip member, the pair of first grooves are farther away from the proximal housing than the pair of second grooves. The design of the different grooves enables eye protection assemblies having different thicknesses to be selectively installed in the clip member to provide more eye protection options for a user's eyes.

In an embodiment, the eye protection assembly comprises at least one of an auto-darkness filter, a welding protective lens with a constant transmittance and a laser protective sheet.

In an embodiment, the side wall of the flip member at least partially protrudes into the distal housing when the clip member is in the close position. The protrusion of the side wall of the clip member into the distal housing guarantees the leakproofness of the entire flexible welding helmet such that arc-light or spatters, occurring during welding works, will be prevented from entering the viewing window assembly via the clip member and thus damaging a user's eyes.

In an embodiment, the belt has an adjustable length.

In an embodiment, the belt comprises two belts, each of which is connected to a respective left or right free edge of the flexible protection shield at one end and releasably connected to the other belt via a buckle at the other end.

In an embodiment, the belt comprises two belts whose length is adjustable, a first belt of the two belts is connected adjacent to a left free end and a right free end of the flexible protection shield respectively, a second belt of the two belts at one end thereof is connected to a top of the flexible protection shield when the flexible welding helmet is worn, and at the other end thereof is connected to the first belt by a belt buckle such that they generate a T-form. According to the present application, the belt(s) can be used to release the load on a user's head when the flexible welding helmet is being worn by the user on his or her head.

In an embodiment, the flexible welding helmet also comprises a fastening feature equipped for the viewing window assembly, by which fastening feature the viewing window assembly can be secured independent of the flexible protection shield when the flexible welding helmet is worn. According to the present application, the viewing window assembly can be secured relative to a user's head by the fastening feature independent of the belt(s). In this way, the load on the user's head can be further released. Therefore, no matter what position the user is taking during his or her work, the viewing window assembly can be always securely kept over the user's face to reliably protect the user's eyes.

In an embodiment, the fastening feature comprises a tightening band which connected to the left and right ends of the proximal edge respectively by two suspensory belts whose length is adjustable.

In an embodiment, a suspensory belt connecting feature is formed at each of the left and right ends of the proximal edge of the proximal housing. Each suspensory belt connecting feature is formed with a connection rod, and the respective one of the suspensory belts is releasably connected to the connection rod by a connection hook such that the suspensory belt is rotatable relative to the connection rod.

In an embodiment, each connection hook is pivotally connected to an additional belt buckle. The respective suspensory belts are connected to the additional buckles. The rotational axis of the connection hook is perpendicular to the pivotal axis of the additional buckle. The combination of the connection hook with the additional buckle enables the position of the suspensory belts to be adjusted in two dimensions such that the viewing window assembly can be more suitably adapted to different facial contours of different users.

In an embodiment, each suspensory belt connecting feature comprises a first suspensory belt connecting part and a second suspensory belt connecting part which are releasably and pivotally connected to each other. The first suspensory belt connecting part is integrally formed on the proximal edge. The connection rod is formed in the second suspensory belt connecting part. The pivotal axis between the first and second suspensory belt connecting parts is perpendicular to the rotational axis of the connection hook.

According to the technical measures of the present application as already mentioned above, the viewing window assembly can be closer to eyes of a user who is wearing the welding helmet on his or her head, such that the visual area of the viewing window assembly through which the user can watch outsides by his or her eyes can be enlarged. Furthermore, the viewing window assembly is designed to substantially follow the face contour of human and the eye shroud of the viewing window assembly can be configured to directly contact the face of the user such that he or she can feel more comfortable.

### BRIEF DESCRIPTION OF THE DRAWINGS

The principles and the other aspects of the present application can be well understood by the following description in combination of the attached drawings. It should be noted that for clarity the drawings might be given here in different ratios, which shall not affect understanding to the description. In the drawings:

FIG. 1 is a perspective view schematically illustrating a flexible welding helmet according to an embodiment of the present application;

FIG. 2 is a perspective view schematically illustrating a flexible welding helmet according to an embodiment of the present application, wherein the flexible welding helmet does not cooperate with a powered air-purifying respirator (PAPR) and is worn on a user's head;

FIG. 3 is an exploded and perspective view schematically illustrating the flexible welding helmet according to the embodiment of the present application;

FIG. 4 is a perspective view schematically illustrating a circlip and a flip member of the flexible welding helmet according to the embodiment of the present application;

FIG. 5A is a perspective view schematically illustrating a viewing window assembly of the flexible welding helmet according to the embodiment of the present application, wherein the flip member of the viewing window assembly is in a close position;

FIG. 5B is a perspective view schematically illustrating the viewing window assembly of the flexible welding helmet according to the embodiment of the present application, wherein the flip member of the viewing window assembly is in an open (or turned-up) position;

FIG. 6A is a perspective view schematically illustrating a bracket which is adopted in the viewing window assembly of the flexible welding helmet according to the embodiment of the present application;

FIG. 6B is a cross-sectional and perspective view schematically illustrating the bracket which is cut out along a line A-A of FIG. 6A;

FIG. 7 is a top view schematically illustrating the viewing window assembly as shown by FIG. 5A;

FIG. 8 is a perspective view schematically illustrating a fastening feature according to an embodiment of the present application for securing the viewing window assembly;

FIG. 9A is a view schematically illustrating a manner in which a belt of the flexible welding helmet according to an embodiment of the present application is connected;

FIG. 9B is a view schematically illustrating another manner in which a belt of the flexible welding helmet according to an embodiment of the present application is connected;

FIG. 10 is a perspective view schematically illustrating a suspensory belt connecting feature according to an embodiment of the present application for securing the viewing window assembly, wherein dashed circles A and B of FIG. 10 are sub-views representing a belt buckle of the suspensory belt connecting feature in different positions respectively;

FIG. 11A is a perspective view schematically illustrating a suspensory belt connecting feature according to another embodiment of the present application for securing the viewing window assembly, wherein for clarity a second suspensory belt connecting part of this suspensory belt connecting feature is separately shown outside the dashed circle;

FIG. 11B is a view schematically illustrating a first suspensory belt connecting feature of the suspensory belt connecting feature in the dashed circle of FIG. 11A; and

FIG. 11C is a view schematically illustrating the first and second suspensory belt connecting features of the suspensory belt structure in the dashed circle of FIG. 11A have been assembled in place.

### DETAILED DESCRIPTION

In the drawings of the present application, those features having the same configuration or having a similar function are represented by the same reference numerals respectively.

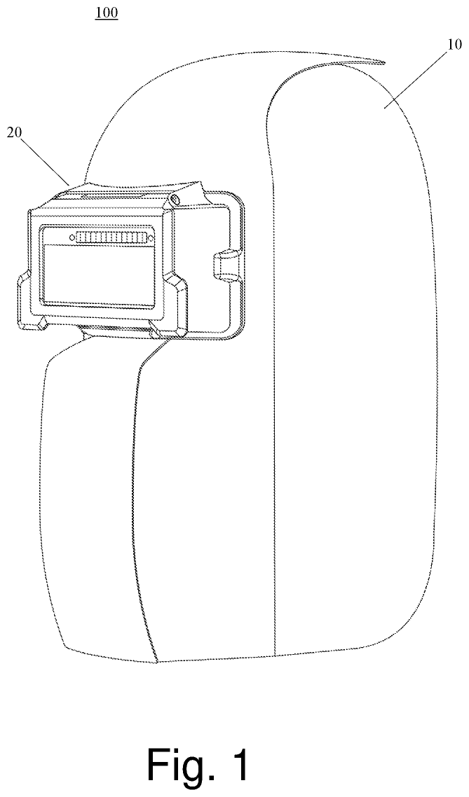

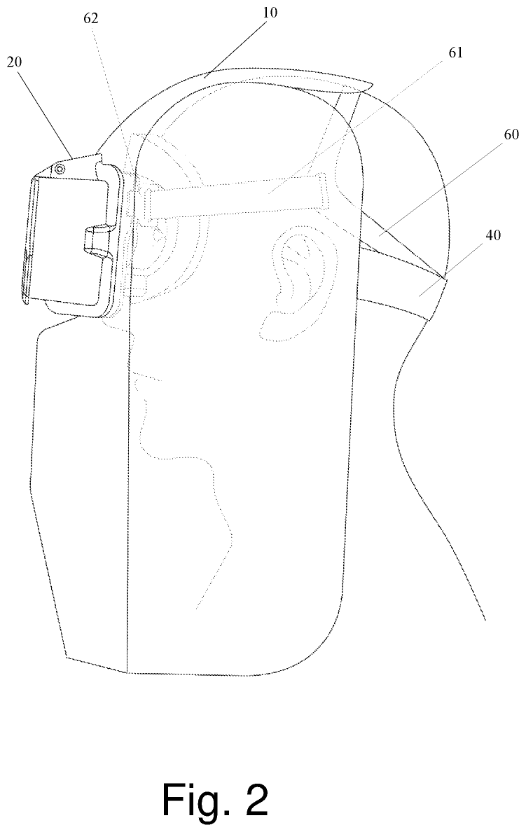

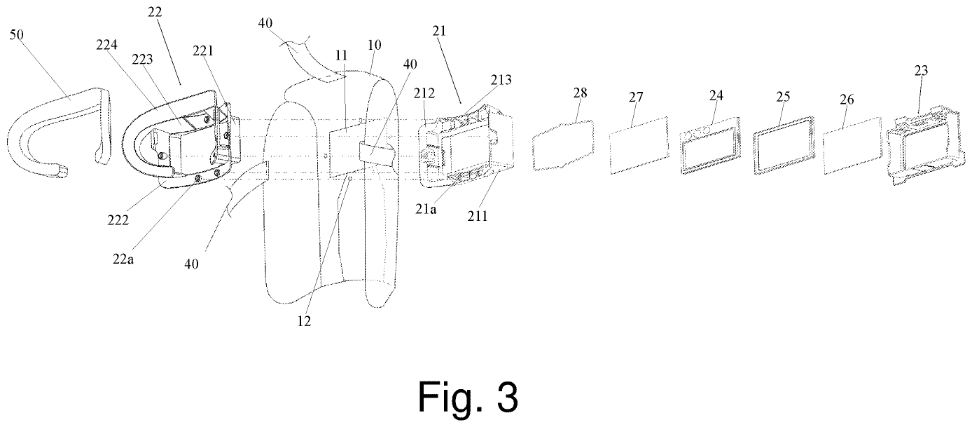

FIG. 1 schematically shows a flexible welding helmet 100 according to an embodiment of the present application. The welding helmet 100 generally comprises a viewing window assembly 20, a flexible protection shield 10 connected to the viewing window assembly 20, and a belt structure 40 for holding and securing the flexible protection shield 10 relative to a user's head. In an embodiment, the flexible welding helmet 100 also comprises a tightening band 60 (not shown in FIG. 1 but shown in FIGS. 2 and 3) configured to hold and secure the viewing window assembly 20 relate to the head of the user.

FIG. 2 is a perspective views schematically illustrating how the flexible welding helmet 100 is worn on a user's head. In this view, light-colored lines represent parts hidden by the flexible welding helmet 100 and the flexible welding helmet 100 is show not to cooperate with a powered air-purifying respirator.

According to an embodiment of the present application, the flexible protection shield 10 can be made of a foldable flexible material with certain protective properties such as a (synthetic or natural) leather, a fireproofing fabric or the like. For instance, the flexible protection shield 10 can be seamed with several pieces of the foldable flexible material. As shown, after the flexible welding helmet 100 is secured in place relative to a user's head, the viewing window assembly 20 is substantially located adjacent to and in front of eyes of the user. According to an embodiment of the present application, a portion of the flexible protection shield 10 under the viewing window assembly 20 is configured to protrude distally, as shown by FIG. 2.

In the context of the present application, the term “distally” or “distal” refers to a direction or a side of the flexible welding helmet 100 or a portion thereof away from a user's head when the flexible welding helmet 100 is suitably worn on the user's head; correspondingly, the term “proximally” or “proximal” refers to a direction or a side of the flexible welding helmet 100 or a portion thereof close to the user's head when the flexible welding helmet 100 is suitably worn on the user's head.

Because the portion of the flexible protection shield 10 under the viewing window assembly 20 is configured to protrude distally, the distally protruding portion of the flexible protection shield 10 is ready for receiving an additional device worn by a user when the flexible welding helmet 100 is worn by the user.

In an alternative embodiment of the present application, the portion of the flexible protection shield 10 under the viewing window assembly 20 can be configured to be planar (i.e. not protrude distally), resulting in a reduction of the total weight of the flexible welding helmet and decreasing the burden on the user's head when the flexible welding helmet 100 is worn by the user.

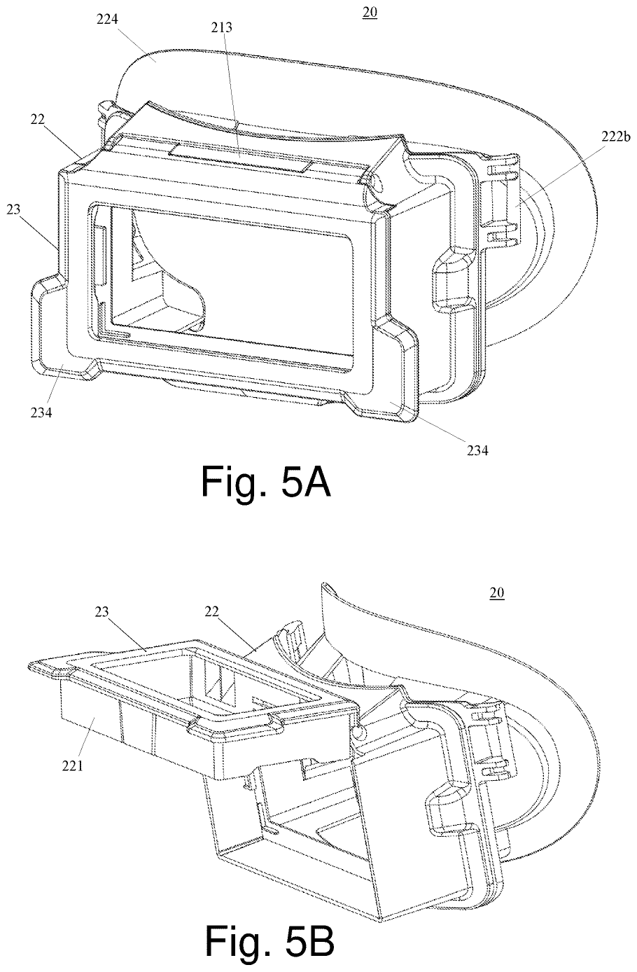

According to an embodiment of the present application, the viewing window assembly 20 is configured to enable eyes of a user to see outsides through the viewing window assembly 20 when the flexible welding helmet 100 is worn in place on the user's head. A shown by FIGS. 3, 5A, and 5B, the viewing window assembly 20 comprises a distal housing 21, a proximal housing 22, and a flip member 23 pivotally connected to the distal housing 21. A plurality of receiving holes 21a are formed in the distal housing 21, and a plurality of through holes 22a are formed in the proximal housing 22. An opening 11 is formed in the flexible protection shield 10, and has a shape substantially corresponding to (or slightly smaller than) a hollow space of either of the distal housing 21 and the proximal housing 22. Moreover, a plurality of through holes 12 are formed adjacent to a circumferential edge of the opening 11. Furthermore, the distal housing 21 is formed with a contact edge 212 and a side wall 211 which extends distally relative to the contact edge 212. The side wall 211 is configured to mainly define the hollow space of the distal housing 21. The contact edge 212 is configured to contact the flexible protection shield 10. The proximal housing 22 is formed with a protrusion wall 221, a contact edge 222 surrounding the protrusion wall 221, a partition wall 223 extending from an inner side of the contact edge 222, and a proximal edge 224 which is connected to the partition wall 223 and is separated from the contact edge 222 by the partition wall 223. As a non-limiting example, the protrusion wall 221 and the contact edge 222 are configured such that they surround the hollow space of the proximal housing 22 completely. The proximal edge 224 is provided in the form of a curved shape with a notch. The notch of the shape is oriented downwards when the flexible welding helmet 100 is worn by the user, such that the curved shape can surround and be adjacent to the bridge of the nose of the user. According to a preferred embodiment of the present application, the proximal edge 222 is provided in a C-shape, wherein the notch of the C-shape is oriented towards and surrounds the bridge of the nose of the user when the flexible welding helmet is worn by the user.

When being assembled, the contact edge 222 of the proximal housing 22 is enabled to contact the flexible protection shield 10 such that the protrusion wall 221 passes the opening 11 of the flexible protection shield 10 and the through holes 22a of the proximal housing 22 align with the through holes 12 of the flexible protection shield 10 respectively. In the meanwhile, the contact edge 212 of the distal housing 21 is enabled to contact the flexible protection shield 10 such that the flexible protection shield 10 is clamped between the distal housing 21 and the proximal housing 22, the protrusion wall 221 of the proximal housing 22 protrudes into the hollow space of the distal housing 21, and the receiving holes 21a of the distal housing 21 align with the though holes 12 of the flexible protection shield 10 and the through holes 22a of the proximal housing 22 respectively. Thereafter, set screws can be engaged into the aligned holes respectively to secure both of the proximal housing 22 and the distal housing 21 together with the flexible protection shield 10.

In a not-shown alternative embodiment, the proximal housing 22, the distal housing 21, and the flexible protection shield 10 can be secured together in any other suitable manner. For instance, the proximal housing 22, the distal housing 21, and the flexible protection shield 10 can be bonded together. For instance, the proximal housing 22 and the distal housing 21 can be snapped into each other along the circumferential edge of the opening 11 of the flexible protection shield 10 such that the flexible protection shield 10 can be clamped between the proximal housing 22 and the distal housing 21.

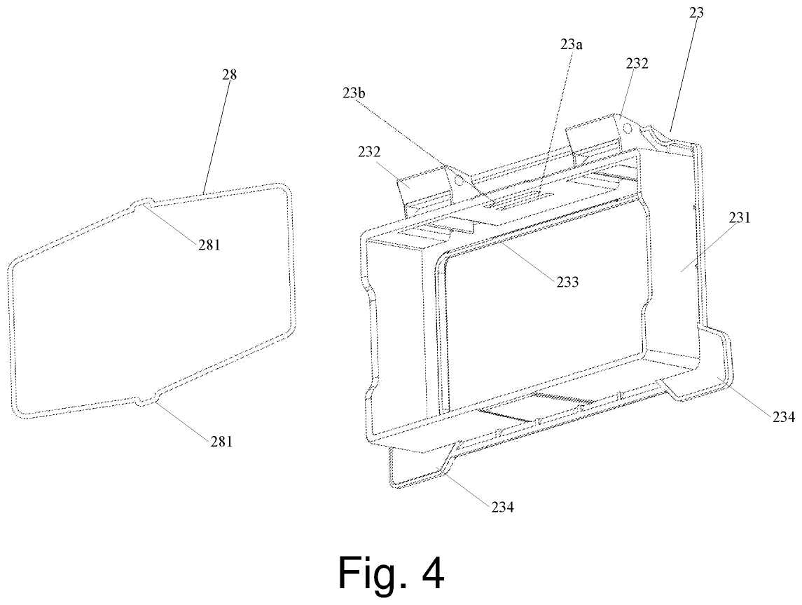

As shown by FIG. 4, the flip member 23 is formed with a side wall 231 to define a hollow space of the flip member 23. A pivotal feature 232 is formed on the side wall 231. For example, the pivotal feature 232 comprises two pivotal features 232. Correspondingly, mating pivotal features 213 are formed on the side wall 211 of the distal housing 21 adjacent to a distal free end of the distal housing 21. For example, the two pivotal features 232 can be installed such that the mating pivotal feature 213 of the distal housing 21 is aligned between the mating pivotal features 213 of the distal housing 21 such that a pivotal shaft passes therethrough to enable the flip member 23 to be pivotally connected to the distal housing 211.

According to a non-limiting example of the present application, after the assembled flexible welding helmet 100 is worn by a user, the two pivotal features 232 of the flip member 23 locate on a top portion of the side wall 231 such that the flip member 23 can reliably rest against the distal free end of the distal housing 21 by gravity. Therefore, the flip member 23 is in a close position relative to the distal housing 21, as shown by FIG. 5A. As a non-limiting example, in the close position, the side wall 231 of the flip member 23 can at least partially protrude into the hollow space of the distal housing 21 such that the side wall 231 at least partially coincides with the protrusion wall 221 and thus arc-light or spatters (which may exist during welding works) can be prevented from entering the distal housing 21 via a gap (if possible) between the two walls. If necessary, the flip member 23 can be lifted up by the user such that the flip member 23 can be pivoted to a turned-up position, as shown by FIG. 5B, in which the user is ready for observing outsides by his or her naked eyes. In order to facilitate the user pivoting the flip member, a lug 234 is formed on the flip member 23. For example, two lugs 234 are shown to be formed on the flip member. Even if in the close position, the lugs 234 is configured to be readily accessible by the user.

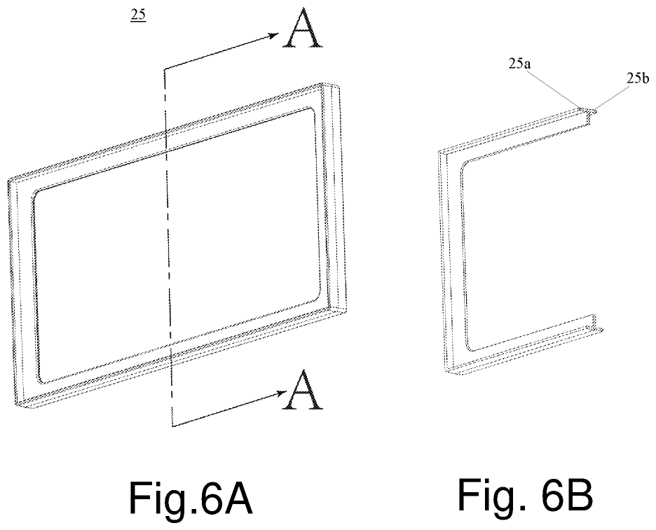

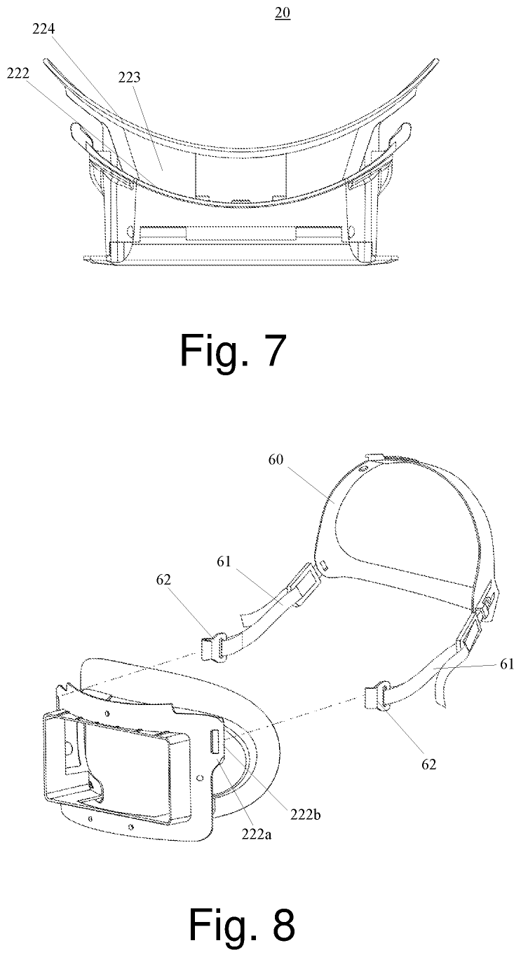

According to an embodiment of the present application, an eye protection assembly is releasably installed in the flip member 23. As a non-limiting example, the eye protection assembly of the present application comprises an auto-darkening filter 24. For instance, the auto-darkening filter 24 can be any suitable auto-darkening filter available in the market which can be automatically darkened to protect eyes of a user during welding works. In an alternative embodiment, the auto-darkening filter 24 can be replaced with a welding protective lens with a constant transmittance. In an alternative or additional embodiment, besides the auto-darkening filter 24 or the welding protective lens with the constant transmittance, the eye protection assembly of the present application can also comprise a laser protective sheet located distally such that the eyes of the user can be protected from damage in the case of laser welding. For example, the auto-darkening filter 24 can be installed in a bracket 25. As shown by FIGS. 6A and 6B, the bracket 25 has a T-shaped cross-section such that the auto-darkening filter 24 in the bracket 25 is surrounded by a circumferential edge 25a of the bracket 25. Furthermore, on an opposing side of the bracket 25 (relative to the auto-darkening filter 24), a distal transparent protective sheet 26 is surrounded by an opposite circumferential edge 25b of the bracket 25. Additionally, a proximal transparent protective sheet 27 can be additionally attached over the auto-darkening filter 24 and surrounded by the circumferential edge 25a of the bracket 25. A groove is formed in the side wall 231 of the flip member 23. For instance, the groove can comprise a pair of first grooves 23a and a pair of second grooves 23b. The first grooves 23a of the pair are formed in the side wall 23 such that they oppose each other, and the second grooves 23b of the pair are formed in the side wall 23 such that they oppose each other. In FIG. 4, only one of the first grooves 23a of the pair is visible and the other is hidden; and only one of the second grooves 23b of the pair is visible and the other is hidden. When the flip member 23 is in the close position, a plane where the first grooves 23a of the pair locate is farther away from the proximal housing 22 than a plane where the second grooves 23b of the pair locate.

A circlip 28 is also installed in the flip member 23. The circlip 28 is configured to removably hold the bracket 25 together with the auto-darkening filter 24, the distal transparent protective sheet 26 and the proximal transparent protective sheet 27 in the flip member 23. For instance, the circlip 28 is formed by a substantially rectangular frame made of an elastic metal wire. Besides, an area surrounded by the rectangular frame is smaller or slightly smaller than an area occupied by the proximal transparent protective sheet 27 and/or the auto-darkening filter 24. Furthermore, the rectangular frame has two opposite bulges 281. The two opposite bulges 281 are configured such that they can be received in the first grooves 23a of the pair or the second grooves 23b of the pair respectively. In the illustrated embodiment, the flip member 23 is formed with a free flange 233. An open area surrounded by the free flange 233 is smaller or slightly smaller than the area occupied by the bracket 25, such that the bracket 25 together with the auto-darkening filter 24, the distal transparent protective sheet 26 and the proximal transparent protective sheet 27 can rest against the free flange 233. Thereafter, the circlip 28 can be snapped into the second grooves 23b of the pair by its bulges 281 respectively. A distance (i.e., an axial distance) of the second grooves 23b from the free flange 233 is designed such that when the circlip 28 is snapped into the second grooves 23b of the pair by the bulges 281 respectively, the circlip 28 is in contact with the proximal transparent protective sheet 27 to axially secure the bracket 25 together with the auto-darkening filter 24, the distal transparent protective sheet 26 and the proximal transparent protective sheet 27 in the flip member 23. In this way, no matter how the flip member 23 is pivoted relative to the distal housing 21, the bracket 25 and the auto-darkening filter 24 can be kept immovable relative to the flip member 23.

The first grooves 23a and the second grooves 23b in the flip member 23 are designed to meet requirements that eye protection assemblies of different thicknesses can be selectively received in the flip member 23. For instance, according to site protection requirements, when an eye protection assembly is embodied as a welding protective lens with a constant transmittance only, the eye protection assembly will have a smaller thickness. In this case, after the welding protective lens with the constant transmittance is installed in the clip member 23, the circlip 28 can be snapped into the first grooves 23 of the pair by the bulges 281 respectively such that the circlip 28 can be used to secure the welding protective lens with the constant transmittance (as the eye protection assembly) in the clip member 23. Therefore, the design of the first grooves 23a and the second grooves 23b guarantees that the eye protection assemblies having different thicknesses can be selectively held in the clip member 23.

According to an embodiment of the present application, the contact edge 212 of the distal housing 21 and the contact edge 222 of the proximal housing 22 are formed such that when the flexible welding helmet 100 is suitably worn, the contact edges 212 and 222 look curved when viewed from above and thus they more closely follow the contour of a user's face or a region from the user's forehead to a part adjacent to the user's nose. Furthermore, the proximal edge 224 of the proximal housing 22 is also shaped to be curved (for example, when the flexible welding helmet 100 is suitably worn, the proximal edge 224 looks curved when viewed from above) to more closely follow the contour of the user's face or the region from the user's forehead to the part adjacent to the user's nose. The notch of the C-shape of the proximal edge leaves a space sufficient for containing the bridge of the nose of the user. According to an embodiment of the present application, the curved C-shaped proximal edge 224 is spaced from the contact edge 222 such that when the flexible welding helmet 100 is worn by the user on his or her head, the contact edge 222 can be closer to the user's nose or the auto-darkening filter 24 can be closer to the user's eyes in the close position of the flip member 23. This will increase the visual area of the viewing window assembly through which the user can observe outsides. In the context of the present application, the term “curved C-shaped” means that the C-shape is not in a planar plane or is not parallel to a planar plane, and in fact a plane where the C-shape locates is curved.

According to an embodiment of the present application, a C-shaped eye shroud 50 made of a soft material or a soft sweat-absorbent material can be additionally provided on the proximal edge 224 of the proximal housing 22. For instance, the C-shaped eye shroud 50 can be bonded onto the proximal edge 224 by a double-sided tape or a Velcro tape. The eye shroud 50 is configured such that its shape at least partially follows that of the proximal edge 224 and thus corresponds to the contour of a user's face or a region from the user's forehead to a part adjacent to the user's nose. Moreover, the notch of the C-shape leaves a space sufficient for receiving the bridge of the nose of the user. Because the viewing window assembly 20 of the present application is in contact with the face of the user via the eye shroud 50, the user can feel more comfortable. In the meanwhile, because the C-shaped shroud 50 is sweat-absorbent, the user will be prevented from feeling uncomfortable due to sweating when the flexible welding helmet 100 is suitably worn.

According to an embodiment of the present application, the partition wall 223 is discontinuously formed between the proximal edge 224 and the contact edge 222 of the proximal housing 22. One or more vent holes are formed in the partition wall 223 for air passing therethrough. The existence of the partition wall 223 enables a gap to be left between the flexible protection shield 10 and a user's face when the flexible welding helmet 100 is worn by the user on his or her head. This discontinuously formed partition wall 223 (or in other words the partition wall 223 formed with the vent holes) enables air to flow smoothly between the flexible protection shield 10 and the face of the user to alleviate dis-comfortable feeling of the user.

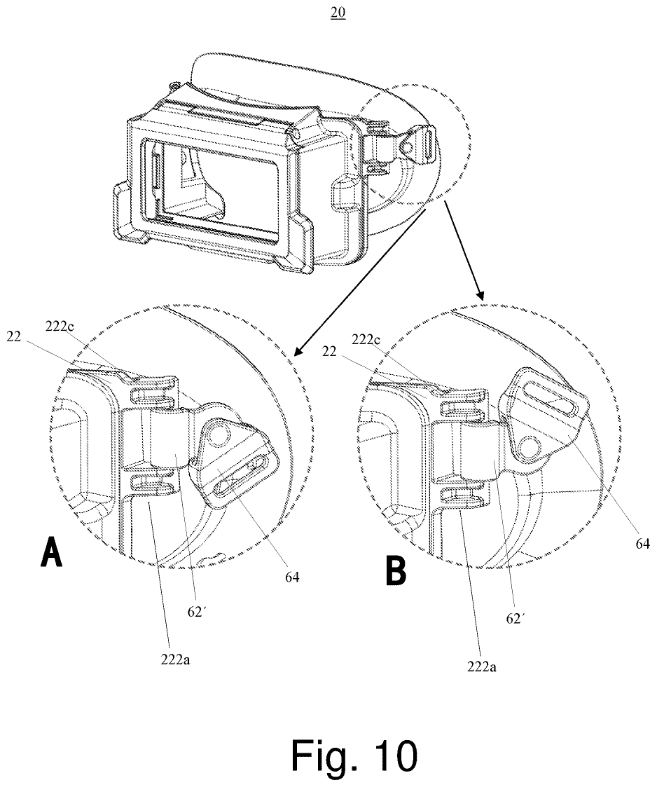

According to an embodiment of the present application, as shown by FIG. 2, the flexible protection shield 10 can be configured to cover both the face and the top of the head of a user. According to an alternative embodiment of the present application, the flexible protection shield 10 can be configured to cover the face of the user only. FIG. 9A schematically illustrates how a belt 40 according to an embodiment of the present application is connected to the flexible protection shield 10. In this embodiment, the belt 40 comprises two belts 40, one of which is connected to a left side (especially its free edge) of the flexible protection shield 10 and the other of which is connected to a right side (especially its free edge) of the flexible protection shield 10. The two belts are releasably connected to each other by a belt buckle at the back side of the user's head. Each belt 40 is configured such that its length is adjustable. FIG. 9B schematically illustrates how a belt 40 according to another embodiment of the present application is connected to the flexible protection shield 10. In this embodiment, one belt 40 is connected to left and right sides of the flexible protection shield 10 at its both ends respectively, one end of the other belt 40 is connected to a part of the flexible protection shield 10 at the top of the user's head, and the other end of the other belt 40 is connected to the one belt 40 to generate a T-form. According to the embodiment of FIG. 9B where the belts 40 are connected when the flexible welding helmet 100 is worn by the user, the load of his or her head can be released.

Normally, when the flexible welding helmet 100 is secured to a user's head via its belt(s) 40, the proximal edge 224 or the eye shroud 50 of the viewing window assembly 20 can naturally seat against the face of the user by gravity.

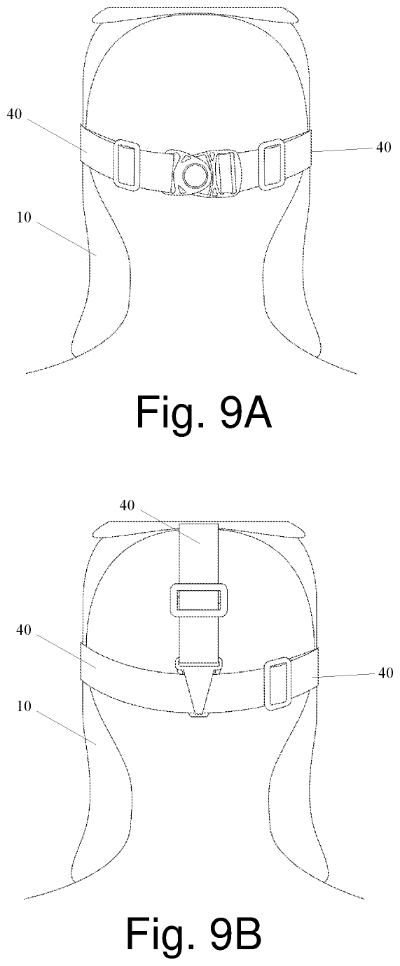

However, in some welding scenarios, it might be required that the viewing window assembly 20 is always kept to reliably contact a user's face to protect his or her eyes no matter what position the user who is wearing the flexible welding helmet 100 on his or her head is taking. In this case, it is required to equip the viewing window assembly 20 with an independent fastening feature. FIG. 8 schematically illustrates one embodiment of the fastening feature. According to this embodiment, the fastening feature comprises a tightening band 60. For example, the tightening band 60 can be made of a soft material such as a plastic material. The tightening band 60 is shaped to be substantially annular and is configured to seat against the back side of the user's head. Two suspensory belts 61 are releasably connected to opposing (left and right) parts of the tightening band 60 respectively. For instance, each suspensory belt 61 is configured such that its length is adjustable. Each suspensory belt 61 is provided with a belt buckle at an end thereof to be connected to the tightening band 60, by which belt buckle the suspensory belt is releasably connected to the tightening band 60. The belt buckle can be any suitable belt buckle well-known by a person skilled in the art.

Two suspensory belt connecting features 222a (only one of which is visible in FIG. 8) are integrally formed on the contact edge 222 of the proximal housing 22 such that they extend laterally outwards from the contact edge. In the illustrated embodiment, each suspensory belt connecting feature 222a is hollowed-out with a connection rod 222b. A free end of each suspensory belt 61 opposing the respective buckle is provided with a connection hook 62. For instance, the connection hook 62 is configured to be releasably locked (for example, hooked) to the connection rod 222b of the respective suspensory belt connecting feature 222a. To this end, the connection hook 62 is configured such that after it is hooked to the connection rod 222b, the connection hook 62 is freely rotatable relative to the connection rod 222b. As shown by FIG. 2, a user can first secure the viewing window assembly 20 onto his or her face via the tightening band 60 and the suspensory belts 61, and then secure the flexible protection shield 10 onto the user's head via the belt(s) 40. In this way, no matter what position the user is taking, the viewing window assembly 20 can be always reliably secured relative to eyes of the user to protect him or her.

According to the present application, after the flexible welding helmet 100 is worn by a user, the connection hooks 62 and the suspensory belt connecting features 222a are sheltered by the flexible protection shield 10 such that they are prevented from being damaged by spatters in welding sites. Additionally, because the contact edge 222 is spaced from the proximal edge 224, the connection hooks 62 will not in contact with the user's face to avoid dis-comfortable feeling of him or her. The level of tightness of the viewing window assembly 20 secured on the face of the user can be adjusted by changing the length of the respective suspensory belt 61.

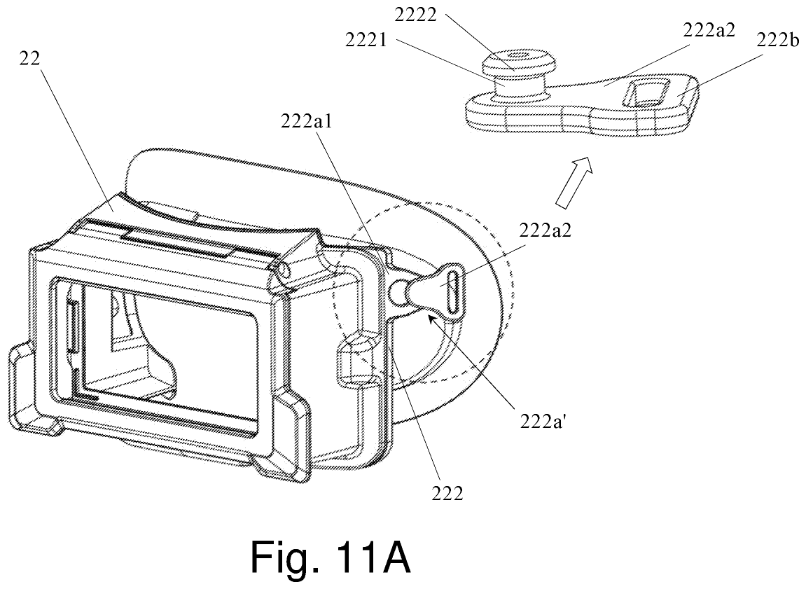

FIG. 10 schematically illustrates a fastening feature according to another embodiment of the present application for securing the viewing window assembly. According to this embodiment, two suspensory belt connecting features 222a (only one of which is visible in FIG. 10) are formed on the contact edge 222 of the proximal housing 22, and each of the suspensory belt connecting features 222a is formed with several reinforcement ridges 222c to increase the strength of the viewing window assembly 20 and thus its securement. Also, each of the suspensory belt connecting features 222a is provided with a connection rod (hidden in FIG. 10). Furthermore, the fastening feature as shown by FIG. 10 also comprises a tightening band 60 and suspensory belts 61 as shown by FIG. 8. However, different than the embodiment of FIG. 8, each of the suspensory belt connecting features 222a in the embodiment of FIG. 10 comprises a connection hook 62′ and an additional belt buckle 64 pivotally connected to the connection hook 62′. The connection hook 62′ is configured in a manner substantially similar to the connection hook 62. The additional belt buckle 64 is designed such that it is pivotable relative to the connection hook 62′ about an axis which is substantially perpendicular to the rotation axis between the connection hook 62′ and the respective connection rod. Therefore, the position of the respective suspensory belt 61 connected to the contact edge 222 of the proximal housing 22 via the respective connection hook 62′ and the respective additional belt buckle 64 is adjustable in two dimensions, so as to be adapted to a user's facial contour such that the flexible welding helmet can be more comfortably worn by the user.

According to another embodiment of the present application, the tightening band 60 is designed such that it can be connected to the flexible protection shield 10 by a Velcro feature. For example, the tightening band can be so connected at the top of a user's head and/or adjacent to the back side of the user's head.

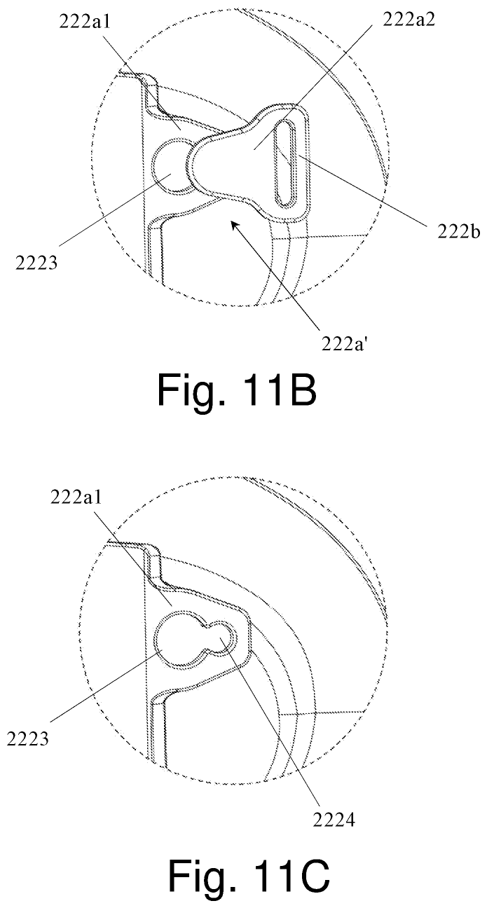

FIGS. 11A to 11C schematically illustrates suspensory belt connecting features according to another embodiment of the present application for securing the viewing window assembly. According to this embodiment, a left suspensory belt connecting feature 222′ and a right suspensory belt connecting feature 222′ are provided on the contact edge 222 of the proximal housing 22 (only one of which suspensory belt connecting features is visible in FIG. 11A). In the embodiment of FIG. 11A, each suspensory belt connecting feature 222′ comprises a first suspensory belt connecting part 222a1 and a second suspensory belt connecting part 222a2. The first suspensory belt connecting part 222a1 is integrally formed at a respective end of the proximal edge 224 of the proximal housing 22. Further as shown by FIG. 11B, a larger circle hole 2223 and a smaller circle hole 2224 are formed in the first suspensory belt connecting part 222a1 such that they communicate with each other and partially coincide with each other. A vertical pivotal pin 2221 is formed in the second suspensory belt connecting part 222a2. The vertical pivotal pin 2221 has an outer diameter substantially equal to the diameter of the smaller circle hole 2224. Further, a circular head 2222 is formed at a free end of the vertical pivotal pin 2221. The circular head 2222 has a diameter which is substantially equal to the diameter of the larger circular hole 2223 and is less than the diameter of the smaller circular hole 2224. In this way, when assembling, the head 2224 of the second suspensory belt connecting part 222a2 is first passed through the larger circular hole 2223 of the first suspensory belt connecting part 222a1, and then the vertical pivotal pin 2221 is pulled into the smaller circular hole 2224 such that the second suspensory belt connecting part 222a2 is kept to be pivotable relative to the first second suspensory belt connecting part 222a1. In this case, the pivotal shaft between the two parts is defined by the pivotal pin 2221 rotatable in the smaller circular hole 2224. Furthermore, the second suspensory belt connecting part 222a2 is formed with a connection rod 222b which is configured to receive the connection hook 62 of FIG. 8. Therefore, in this embodiment, the suspensory belt connecting features also allow the position of the respective suspensory belt 61 to be adjustable in two dimensions due to the existence of the connection hooks 62, such that the flexible welding helmet can be adapted to a user's facial contour and thus the flexible welding helmet can be more comfortably worn by the user. Furthermore, it should be understood by a person skilled in the art that any other suitable structure can be adopted in the technical solution of the present application as soon as the structure can be used to releasably connect the first and second suspensory belt connecting parts 222a1 and 222a2 such that they are pivotable relative to each other. For example, the first and second suspensory belt connecting parts 222a1 and 222a2 can be connected to each other by a pivotal pin releasably passing both of them. In summary, the pivotal axis between the first and second suspensory belt connecting parts 222a1 and 222a2 is not coplanar with the pivotal axis of the respective connection hook 62 or even is substantially perpendicular to the same.

Although some specific embodiments of the present application have been described here for illustrative purposes only, they cannot be deemed to constrain the claim scope of the present application. Furthermore, it should be understood by a person skilled in the art that the embodiments and examples mentioned in the description can be combined with each other. Without departing from the spirit and scope of the present application, various replacements, modification and alternations can be thought out.

1. A flexible welding helmet, comprising:
a viewing window assembly;
a flexible protection shield releasably connected to the viewing window assembly; and
a belt connected to the flexible protection shield and having an adjustable length, wherein the viewing window assembly comprises:
a distal housing, a proximal housing, and a flip member pivotally connected to the distal housing, the distal housing is provided with a contact edge, the proximal housing is provided with a contact edge and a proximal edge spaced from the contact edge, the flexible protection shield is partially clamped between the contact edge of the distal housing and the contact edge of the proximal housing, the proximal edge of the proximal housing has a curved contour which has a notch, the notch is configured to direct downwards when the flexible welding helmet is worn, and an eye protection assembly is releasably installed in the flip member.

2. The flexible welding helmet according to claim 1, wherein the proximal edge of the proximal housing is curved C-shaped such that opposing left and right ends of the proximal edge are farther away from the flip member than a middle portion of the proximal edge, and the notch of the C-shape directs downwards when the flexible welding helmet is worn.

3. The flexible welding helmet according to claim 2, wherein a partition wall is formed between the contact edge of the proximal housing and the proximal edge of the proximal housing, and at least one aperture is formed in the partition wall.

4. flexible welding helmet according to claim 3, wherein an eye shroud made of a soft material or a soft sweat-absorbent material is releasably installed on the proximal edge of the proximal housing, and the eye shroud is shaped to follow the proximal edge.

5. The flexible welding helmet according to claim 4, wherein it also comprises a fastening feature equipped for the viewing window assembly, by which fastening feature the viewing window assembly can be secured independent of the flexible protection shield when the flexible welding helmet is worn.

6. The flexible welding helmet according to claim 5, wherein the fastening feature comprises a tightening band which connected to left and right ends of the proximal edge respectively by two suspensory belts whose length is adjustable.

7. The flexible welding helmet according to claim 6, wherein a suspensory belt connecting feature is respectively formed at each of the left and right ends of the proximal edge of the proximal housing, each suspensory belt connecting feature is formed with a connection rod, and a respective one of the suspensory belts is releasably connected to the connection rod by a connection hook such that the respective suspensory belt is rotatable relative to the connection rod.

8. The flexible welding helmet according to claim 7, wherein each connection hook is pivotally connected to an additional belt buckle, the respective suspensory belt is connected to the additional belt buckle, and a rotational axis of the connection hook is perpendicular to a pivotal axis of the additional belt buckle.

9. The flexible welding helmet according to claim 7, wherein each suspensory belt connecting feature comprises a first suspensory belt connecting part and a second suspensory belt connecting part which are releasably and pivotally connected to each other, the first suspensory belt connecting part is integrally formed on the proximal edge, the connection rod is formed in the second suspensory belt connecting part, and a pivotal axis between the first and second suspensory belt connecting parts is perpendicular to a rotational axis of the connection hook.

10. The flexible welding helmet according to claim 9, wherein the contact edge of the proximal housing and the contact edge of the distal housing are curved such that opposing left and right ends of the contact edge are farther away from the flip member than a middle portion of the contact edge.

11. The flexible welding helmet according to claim 10, wherein the flexible protection shield has a part which locates under the viewing window assembly when the flexible welding helmet is worn and which is configured to protrude distally or be planar.

12. The flexible welding helmet according to claim 11, wherein the flip member is pivotable between a close position and a turned-up position, in which close position, the eye protection assembly covers a hollow interior space of the viewing window assembly defined by the distal housing and the proximal housing, and in which turned-up position, the eye protection assembly uncovers the hollow interior space.

13. The flexible welding helmet according to claim 12, wherein the flip member is formed with a side wall to define a space in which the eye protection assembly is installed.

14. The flexible welding helmet according to claim 13, wherein at least one pair of grooves are formed in the side wall of the flip member, the grooves of the pair are configured to oppose each other to receive bulges of a circlip installed in the space of the flip member, the circlip is configured to contact the eye protection assembly to secure it within the space of the flip member, the at least one pair of grooves include a pair of first grooves and a pair of second grooves, in the close position of the flip member, the pair of first grooves are farther away from the proximal housing than the pair of second grooves.

15. The flexible welding helmet according to claim 14, wherein the eye protection assembly comprises at least one of an auto-darkness filter, a welding protective lens with a constant transmittance and a laser protective sheet.

16. The flexible welding helmet according to claim 15, wherein the belt comprises two belts, each of which is connected to a respective left or right free edge of the flexible protection shield at one end and releasably connected to the other belt via a buckle at the other end.

17. The flexible welding helmet according to claim 15, wherein the belt comprises two belts whose length is adjustable, a first belt of the two belts is connected adjacent to a left free end and a right free end of the flexible protection shield, a second belt of the two belts at one end thereof is connected to a top of the flexible protection shield when the flexible welding helmet is worn, and at the other end thereof is connected to the first belt by a belt buckle such that they generate a T-form.
